(19) United States
(12) Patent Application Publication
(10) Pub. No.: US 2026/0144722 A1
(43) Pub. Date: May 28, 2026

(54) Nail Lacquer

(71) Applicant: JBRD S.A., Luxembourg (LU)

(72) Inventors: Jean-Raymond Boulle, Luxembourg (LU); Hans Halse Hornemann, Luxembourg (LU); Jasmin Patzschke, Luxembourg (LU)

(73) Assignee: JBRD S.A., Luxembourg (LU)

(21) Appl. No.: 19/164,522

(22) PCT Filed: Mar. 1, 2024
(86) PCT No.: PCT/EP2024/055493

§ 371 Date: Sep. 11, 2025

(30) Foreign Application Priority Data
Mar. 3, 2023 (GB) 2303129.7
Mar. 1, 2024 (WO) PCT/EP2024/055493

(51) Int. Cl. A61K 8/19; A61K 8/73

(52) U.S. Cl. CPC A61K 8/19; A61K 8/731; A61K 2800/30; A61K 2800/43

(57) ABSTRACT

The present invention relates to the field of human or animal nailcare, and relates in particular to nail lacquers which have safe properties and/or contain a particular distribution of diamond particles. The inventive lacquer is preferably composed of substances generally considered to be safe (also known in the field as GRAS—Generally Regarded As Safe). By lacquer we mean any liquid to be applied to a nail and which sets to form a hard film or layer. The invention provides a liquid lacquer composition for forming a solid cosmetic coating on nail tissue, the composition comprising: one or more film-forming polymers or polymer precursors, one or more solvent for the polymer or polymer precursor, one or more plasticizers which promote flexibility of the solid cosmetic coating and optionally one or more pigments or optical modifiers. The liquid lacquer composition preferably further comprises diamond particles having a mean size within the range of 0.1 micron to 10 microns dispersed within the liquid. The diamonds may be present in amounts of 0.01 to 0.20 wt %.

### FIELD OF THE INVENTION

The present invention relates to the field of human nailcare, and relates in particular to nail lacquers which have safe properties and/or contain a particular distribution of diamond particles. The inventive lacquer is preferably composed of substances generally considered to be safe (also known in the field as GRAS—Generally Regarded As Safe). By nail lacquer we mean any liquid to be applied to a human or animal nail and which sets to form a hard film or layer. This includes nail polishes which rely upon air-drying or evaporation of a solvent, or those having two or more components which react to produce the solid lacquer. Nail lacquers may be applied in distinct layers, typically a base coat, a pigmented nail polish layer and a gloss top coat. The base cost is applied directly to the nail tissue and is thus formulated to provide good direct adhesion. The nail polish layer is usually intended to provide a colour or light effects such as pearlescence or visible reflective effects. The top gloss coat serves to protect the nail polish and to provide a gloss finish. In practice the layers each have similar formulations. In some consumer nail polishes, a single nail polish application may take place and provide an adequate coloured film, with high gloss and hardness.

### BACKGROUND OF THE INVENTION

Nail polishes are products used on nail tissue, primarily for aesthetic reasons. Nail polish has been known for several thousand years and was originally based on mixtures of beeswax, egg whites, gelatin, vegetable dyes, and gum Arabic all of natural origin. Most modern-day state of the art formulations are made from three major types of mainly artificially derived ingredients

### 1. Polymers.

These make up the base ingredients of the polish. These are usually supplied as a solution of nitrocellulose and a modifying agent such as tosylamide/formaldehyde resin (TSF resin). Together they produce a lacquer having a hard, shiny (i.e. glossy) and flexible surface which has strong adhesion to the underlying keratin tissue of the nail. Nitrocellulose-TSF combinations have proved to be effective in commercial formulations, but the resin is facing increased opposition by consumers due to minor content of formaldehyde, which is a known carcinogenic material. Furthermore, there are reports of cases of allergic and skin irritation by users of nail polishes containing the ingredient.

Nitrocellulose polymer is a liquid solution of the solid reaction product of cotton or wood chips being treated with nitric and sulfuric acids and a suitable solvent. The solvents that can be used with nitrocellulose include almost any organic solvent, like acetone and/or ethanol. Tosylamide/formaldehyde is a synthetic film former resin which is added to improve the plasticity and adhesion of the nitrocellulose when applied.

### 2. Plasticizers.

These are added to increase flexibility and durability of the solid lacquer. Among them, camphor has been extensively used as it increases flexibility in the formulations, but this along with Dibutyl Phthalate and other ingredients with similar functionality has been banned in the European Union. Replacements for these ingredients are available but are generally associated with a loss of functionality and aesthetic appearance.

### 3. Pigments.

These may be added to the lacquer formulations to provide colour and other visual effects, such as pearlescent effects.

Over time, some users of nail polish standard formulations have experienced allergic reactions, a general breakdown of the surface keratin layer of the nail leading to a pitted, unsmooth surface and uneven colouration. Hence there is a need for a complete reconsideration and revision of the ingredient palette with an emphasis on robust natural alternatives and retention of the functionality of especially the properties of the solid set lacquer.

U.S. Pat. No. 7,294,340B2 (published on 13 Nov. 2007) discloses the use of nanodiamonds in cosmetics such as nail polish compositions, lip gloss and eyeliner. The nanodiamond particles have an average size that is from 0.5 nm to 50 nm (preferably 0.5 nm to 10 nm). The nail polish uses nitrocellulose resin, with ethyl acetate, toluene, and isopropyl alcohol as solvents, and dibutyl phthalate as plasticizer. Although it is claimed that nanodiamonds provide improved strength (and resistance to chipping and wear), no specific evidence of this is provided. The compositions of this patent application typically include nanodiamond particles in amounts of 1 to 60 wt % of the composition. When incorporated into the lacquer, the nanodiamonds can apparently reinforce the structure of the coating, making it more resistant to scratching, chipping, and general wear and tear.

It is supposed that the small size of nanodiamonds allows them to penetrate deeply into the lacquer matrix, creating a strong and durable composite material. This reinforcement improves the overall hardness of the nail lacquer, providing better protection and longevity for the nails.

Additionally, nanodiamonds can also enhance the gloss and smoothness of the nail lacquer, giving it a more polished and professional appearance. It is essential to ensure that the nanodiamonds used are of high quality, properly dispersed within the lacquer matrix, and compatible with other ingredients to achieve the desired results without compromising product safety or performance.

Overall, incorporating nanodiamonds into the formulation can result in a high-performance nail lacquer that is both durable and aesthetically pleasing.

Nail polish with nanodiamonds is a relatively new innovation in the world of cosmetics.

Nanodiamonds are small particles of diamond, by definition less than 100 nanometers in size. These are therefore not individually resolvable by an observer using the naked eye in visible light. Diamonds can be added to nail polish to create a glittering, sparkling effect but generally the particle size to have an optically visible effect needs to be larger than approximately 74 microns. One of the main benefits of using nail polish with nanodiamonds is that it can help strengthen the lacquer and protect nails. The nanodiamond particles can also help seal in moisture, which may keep nails hydrated and healthy.

However, nail polish with nanodiamonds can be more expensive than traditional nail polish due to the added cost of the diamond particles in the preferred size range and distribution. It has also been observed that there is consumer resistance to the presence of nanoparticles in cosmetic products, hence their mention as an objectionable ingredient in the 21-FREE list per Table 2 above. Whether or not members of the list are harmful or not, consumers are guided by perceived risk, which ultimately dictates whether products are successful in the market. There is therefore a need to provide an alternative method for strengthening nail lacquers so as to improve longevity, scratch resistance and adherence to underlying nails.

The present invention in various aspects seeks to provide lacquer compositions (which may be base coat, nail polish and/or top coats) which use safer and less objectionable ingredients, without compromising performance. It is also an object to provide lacquer compositions which are harder, more scratch resistant and retain good adherence.

The foregoing objectives are met, at least in part, by lacquer compositions in accordance with the present invention as set out below and/or in the claims hereinafter.

### STATEMENTS OF INVENTION

The present invention relates to formulations of a base coat, a nail polish and a top coat. The family of formulations are based on ingredients which are well-documented and proven to be generally considered as safe (GRAS) and retain functionality that ensures top-of-the-class performance concerning longevity, anti-chipping and gloss. The functionality is enhanced by the addition of micro diamond powder that induces the formulations with extra longevity, adhesion and anti-chipping resistance and also compensates for losses of functionality experienced by other nail care products formulated with natural and non-toxic alternatives to the prevailing synthetic, largely petrochemical ingredients. The new ingredient formulations help to repair the underlying nail tissue if damaged and help smooth out minor discolouration and imperfections of the nail surfaces.

In one aspect of the invention the invention provides a liquid lacquer composition for forming a solid cosmetic coating on nail tissue, the composition comprising:
one or more film-forming polymers or polymer precursors,
one or more solvent for the polymer or polymer precursor,
optionally one or more plasticizers which promote flexibility of the solid cosmetic coating and optionally one or more pigments or optical modifiers.

In a preferred aspect of the invention the composition further comprises micron scale diamond particles. These preferably have a mean size within the range of 0.1 micron to 10 microns dispersed within the liquid composition. The diamond particles more preferably have a mean size within the range of 0.2 to 5.0 microns, most preferably within the range of 0.3 to 3.0 microns.

The mean particle size has a standard deviation and the particle size+/−its standard deviation preferably falls within the range. Most preferably the particle size lower Di and upper D99 values fall within the range. This ensures that nanodiamonds and over-size particles are essentially excluded. The invention may provide in one aspect nail compositions in accordance with the invention where diamond nanoparticles are absent.

A dispersant may be provided for the diamond particles, such as fumed silica and/or stearalkonium bentonite. The particles may be added pre-mixed with the dispersant, film forming polymer and solvent.

The diamond particles may be present in an amount of about 0.01 to 0.20 wt %, preferably about 0.02 to 0.08 wt %, and in a most preferred range 0.03 to 0.07 wt %. Amounts outside of these ranges can be used if particular applications demand it.

Compared to nanodiamonds-based liquid formulations, a person skilled in the art would expect micron-sized diamonds to have more difficulty dispersing evenly throughout the lacquer matrix due to their larger size and tendency to agglomerate or settle. The larger size of micron scale particles would make them less able to penetrate the nail surface and therefore less able to provide enhancement of physical properties of the nail or lacquer.

The present inventors have discovered that, unexpectedly, the particles in the size range claimed in accordance with the present invention do in fact provide better strength and durability in nail lacquers. Furthermore, users have provided anecdotal reports of improved nail tissue condition after shifting to the diamond-containing lacquers of the present invention.

In accordance with a further aspect of the invention the lacquer composition is selected to comply with the GRAS A1 or A2 or B standard of the United States Environmental Protection Agency (EPA).

This may require exclusion of certain objectionable, relatively unsafe, compounds or chemicals. So, the polymer/precursor may exclude tosylamide/formaldehyde resin (TSF Resin).

The plasticizer may exclude dibutyl phthalate (DBP), trimethyl pentanyl diisobutyrate, triphenyl phosphate, camphor. The pigment, when present, excludes mica, titanium dioxide, bismuth oxychloride, Cl 777266.

The solvent may exclude propyl acetate, toluene, benzophenone-1, dimethicone.

The film forming polymer may comprise a nitrocellulose. This compound is potentially harmful, but not when included in small amounts in a nail lacquer. The solvent may be selected from one or more of: ethyl acetate, butyl acetate, ethyl alcohol, isopropyl alcohol, stearalkonium bentonite, acetone, methyl ethyl ketone (MEK). These may be provided in amounts so small as to reduce hazard to negligible amounts. The polymer or polymer precursor may be selected from one or more of: adipic acid I neopentyl glycol I trimellitic anhydride copolymer, acrylates copolymer, cellulose acetate butyrate.

The plasticiser may be selected from one or more of: acetyl tributyl citrate, triethyl citrate, and sucrose benzoate.

The pigment may be selected from one or more of: silica, citric acid, ferric ammonium ferrocyanide, D & C Red #6 Barium Lake, D & C Red #7 Calcium Lake, FD & C Yellow #5 Aluminum Lake, Cl 77891, Cl 5880, aluminium hydroxide, tin oxide, Cl 77499, Cl 77007, Cl 77742, Cl 12085, Cl 42090, Cl 77000, Cl 60725.

The invention also provides for a base coat or nail polish or gloss coat for application to human nails, comprising a liquid lacquer in accordance with any of the hereinbefore described lacquer compositions.

### DETAILED DESCRIPTION OF THE INVENTION

Before the application of nail polish in the present context it is assumed that all necessary preparations in the form of cleaning and sanitising the application area have been performed. The subsequent application procedure consists of 2 to 3 application steps, all with polymers dissolved in a solvent, i.e., nail polish systems that harden/solidify through evaporation of the solvents. Systems that require exposure to ultraviolet light to harden are not considered. The application sequence consists of one, two, or all of the following steps:
1) Base coat
2) Nail polish with colour sometimes repeated to ensure full coverage.
3) Top coat to make the application long-lasting, sometimes left out.

“Alustre” is one brand name adopted for the new formulations, some of which are provided with infused micro diamonds. This name is used henceforth in the following text to designate the suite of products, i.e., base coat, nail polish, and top coat, which are compositions in accordance with one or more aspects of the invention.

The ingredient list of the products used in each of the application steps is generally the same, but concentrations are adjusted to optimize the functionality. The general list of ingredients is stated in Table 1 below.

TABLE 1
General Ingredient list for nail polish

| Basecoat/nail polish/top coat | Function |
|---|---|
| ETHYL ACETATE | Solvent |
| BUTYL ACETATE | Solvent |
| NITROCELLULOSE | Film former |
| ALCOHOL | Solvent |
| COPOLYMERS | Polymer modifiers |
| PLASTICISERS | Solvents and film modifiers |
| Other organic and inorganic ingredients | Pigments |

The primary film-forming ingredient used in nail polish remains nitrocellulose, with solvent derived from a fermentation process. Once the solvent evaporates (drying time being an important functional property), the resulting coating can become brittle and stick inadequately to the nails. To ensure good adhesion and flexibility, other polymers or plasticizers are added to the formula. In addition to mechanical functionality, nail polish must also resist exposure to cosmetic oils, soap, water, exposure to sunlight etc. which nails are commonly exposed to in everyday life.

Modern nail polish formulations have unfortunately taken prior inspiration from the range of ingredients also used for industrial paints. However, to avoid the use of toxic ingredients that can cause adverse allergic reactions or other health effects, the formulations presented here are replacing these ingredients with healthier alternatives derived from well-proven natural substances either labelled “Generally Recognized as Safe” (GRAS)—in Table 2 below labelled A1, or “considered a relatively safe chemical for use in industrial, commercial, and consumer applications” which in Table 3, below bears the label A2. The GRAS labelling systems are developed and maintained by the U.S. Environmental Protection Agency (EPA).

The statements are based on a combination of scientific research and regulatory assessments of a particular chemical's safety. This process typically involves multiple stages of testing, including animal studies, human studies, and environmental assessments. The European Chemicals Agency (ECHA) similarly may classify the chemical as safe for certain uses, with appropriate restrictions and guidelines for handling and disposal. Under the Cosmetics Regulation, all cosmetic ingredients must undergo a safety assessment before they can be placed on the market. The safety assessment takes into account the intended use of the cosmetic, the potential exposure to the ingredient, and the available toxicological data on the ingredient. The cosmetic industry itself has created a list of 21 ingredients that are seen as a guideline for unwanted ingredients in cosmetic products.

TABLE 2
21 -Free list originally made by the founder of the nail
polish brand “Priti NYC, named Kim D'Amato
21-FREE list

1. Formaldehyde
2. Toluene
3. Dibutyl phthalate (DBP)
4. Formaldehyde resin
5. Camphor
6. Triphenyl phosphate (TPHP)
7. Xylene
8. Ethyl tosylamide
9. Parabens
10. Acetone
11. Butyl acetate
12. Butylated hydroxyanisole (BHA)
13. Ethanolamine
14. Glycol ether
15. Lead
16. Mercury
17. MEK (Methyl ethyl ketone)
18. Mineral oil
19. Nanoparticles
20. Petrochemicals
21. Gluten

For example, formaldehyde and toluene were once commonly used ingredients in nail polish formulations. These chemicals can still be found in some nail products, so there is a need to phase out these chemicals and shift towards greener and more natural formulations that use alternative, healthier ingredients. Formaldehyde and toluene have been known to cause allergic reactions, such as swollen eyelids, in some people and are classified as carcinogenic.

Studies have shown that the use of nitrocellulose solution in nail polish can have some negative effects on the environment, as it is considered a hazardous waste and can release harmful gases when burned. Hence the ingredient bears the label B in table 3. The amount of nitrocellulose used in nail polish is typically relatively small, as is evident from table 3 and it solidifies in a matter of a few seconds when the appropriate solvent is being used. The hardened nitrocellulose film on nails from nail polish is generally considered safe for most people.

TABLE 3
List of ingredients and concentration levels in
nail products according to the present invention

| | Base | Nail | Top | | Notes of |
|---|---|---|---|---|---|
| | coat | polish | coat | Health | excluded |
| Ingredient | (%) | (%) | (%) | aspect | ingredients |
| Ethyl acetate (CAS # 141-78-6) | <38 | <42 | <36 | A1 | |
| Butyl acetate (CAS # 123-86-4) | <28 | <31 | <35 | A2 | |
| Nitrocellulose (CAS # 9004-70-0) | <11 | <12 | <8.5 | B | |
| Ethyl alcohol (CAS # 64-17-5) | <6 | <6 | <5 | A1 | |
| Isopropyl alcohol (CAS # 67-63-0) | <0.2 | <0.15 | nil | A1 | |
| Polymers | | | | | |
| Tosylamide/Formaldehyde resin | x | x | x | — | 1 |
| Adipic acid/neopentyl glycol/trimellitic | <5.3 | <5.3 | n.p. | A2 | |
| anhydride copolymer | | | | | |
| Acrylates copolymer | <4 | <4 | <4 | A2 | |
| Cellulose acetate butyrate | n.p. | n.p. | <3 | A2 | |
| Plasticizers | | | | | |
| Dibutyl phthalate (DBP) | x | x | x | — | 2 |
| Trimethyl pentanyl diisobutyrate | x | x | x | — | 3 |
| Triphenyl phosphate | x | x | x | — | 4 |
| Camphor | x | x | x | — | 5 |
| Acetyl tributyl citrate | <5 | <5 | <5 | A2 | |
| Triethyl citrate | <5 | <5 | <5 | A2 | |
| Sucrose benzoate | n.p. | <0.2 | n.p. | A1 | |
| Pigments | | | | | |
| Mica | x | x | x | — | 6 |
| Silica | <0.4 | <0.65 | n.p. | A1 | |
| Titanium dioxide | x | x | x | — | 7 |
| Bismuth oxychloride | x | x | x | — | 8 |
| Ferric ammonium ferrocyanide | <0.001 | <0.001 | x | A1 | |
| D&C Red #6 Barium Lake | <0.005 | <1.3 | x | A2 | |
| D&C Red #7 Calcium Lake | x | <0.6 | x | A2 | |
| FD&C Yellow #5 Aluminium Lake | x | <1.4 | x | A2 | |
| CI 777266 | x | x | x | — | 9 |
| CI 77891 | <0.08 | <2.7 | x | A2 | |
| CI 15880 | x | <0.8 | x | A2 | |
| Aluminium hydroxide | <0.1 | <0.1 | x | A2 | |
| Tin oxide | n.p. | <0.02 | n.p. | A2 | |
| CI 77499 | n.p. | <3.0 | n.p. | A2 | |
| CI 77492 | n.p. | <1.0 | n.p. | A2 | |
| CI 77491 | n.p. | <0.8 | n.p. | A2 | |
| CI 42090 | n.p. | <0.5 | n.p. | A2 | |
| CI 77000 | n.p. | <0.06 | n.p. | A2 | |
| CI 60725 | n.p. | 0.002 | n.p. | A2 | |
| Solvents | | | | | |
| Ethyl Alcohol | <6 | <6 | <5 | A1 | |
| Ethyl Acetate | <38 | <42 | <36 | A2 | |
| Propyl acetate | x | x | x | | 10 |
| Butyl Acetate | <28 | <31 | <35 | A2 | |
| Toluene | x | x | x | | 11 |
| Stearalkonium bentonite | <1.5 | <1.7 | x | A2 | |
| Benzophenone-1 | x | x | x | | 12 |
| Dimethicone | x | x | x | | 13 |
| ACETONE | n.p. | <2 | n.p. | | 14 |
| Methyl ethyl ketone (MEK) | n.p | <0.01 | <0.01 | | 15 |

Key:
n.p. = Not present
A1: Substance “Generally Recognized as Safe” (GRAS)
A2: Substance “Considered a relatively safe chemical for use in industrial, commercial, and consumer applications”
B: Substance is generally considered safe, specifically in nail polish because relatively minor amounts are used and after application, the hardened film thus obliterates fumes and solvent.

### Specific Examples and Performance Testing

Initially functional testing was done with the following performance (functional) parameters:
1. Gloss evaluation
2. Drying time
3. Hardness after 24 hrs
4. Adhesion test.

The testing was performed to provide a comparison of Alustre products against seven commercially available brands to assess the 4 main functional parameters.

TABLE 4
Functional parameters of Alustre Nail Polish against market-leading brands

| | Gloss | Drying | | | |
|---|---|---|---|---|---|
| | evaluation | times | Hardness | Adhesion | Notes (loss of |
| Product name | result | recorded | after 24 h | test result | coating material) |
| Alustre 227405607 | 84 | 2′20″, 3′30″ | 176 | 0 | No loss of coating |
| | | | | | material |
| Jinsoon Nail Lacquer | 81 | 5′54″, 7′15″ | 137 | 0 | No loss of coating |
| | | | | | material |
| Nails Inc. Vegan | 79 | 4′13″, 5′05″ | 97 | 1 | Loss of small |
| Polish | | | | | coating materials |
| | | | | | particles |
| Dior Vernis Gel Shine | 67 | 3′36″, 4′30″ | 195 | 0 | No loss of coating |
| & Long Wear Nail | | | | | material |
| Lacquer | | | | | |
| Olive & June 7-Free | 74 | 4′15″, 5′00″ | 148 | 0 | No loss of coating |
| Polish | | | | | material |
| Chanel Le Vernis | 77 | 3′45″, 4′48″ | 183 | 0 | No loss of coating |
| Nail Colour | | | | | material |
| YSL La Laque | 80 | 4′54″, 6′00″ | 180 | 1 | Loss of small |
| Couture | | | | | coating materials |
| | | | | | particles |
| Kure Bazaar | 79 | 6′05″, 7′12″ | 132 | 0 | No loss of coating |
| | | | | | material |

TABLE 5
Functional parameters of Alustre Top
Coat against 7 market-leading brands.

| | Gloss | | Hardness | Solid |
|---|---|---|---|---|
| | evaluation | Drying times | after | content |
| Product name | result | recorded | 24 h | (%) |
| Alustre T227414 | 85 | 1′54″, 2′24″ | 125 | 25.22 |
| Seche Vite Dry Fast | 84 | 2′06″, 3′00″ | 263 | 23.58 |
| Le Mains Hermes | 88 | 3′12″, 4′00″ | 180 | 35.26 |
| Top Coat | | | | |
| TEN OVER TEN The | 86 | 2′00″, 2′54″ | 307 | 18.66 |
| Shield | | | | |
| Dior Top Coat Abricot | 85 | 1′45″, 2′30″ | 276 | 21.47 |
| Hardwear Shine UV Top | 84 | 1′15″, 2′00″ | 297 | 24.48 |
| Coat Butter London | | | | |
| London Town Kur Gel | 88 | 1′12″, 1′54″ | 285 | 20.45 |
| Genius Top Coat | | | | |
| People of Color | 88 | 3′48″, 5′18″ | 103 | 28.73 |

The foregoing results show that the diamond-free, GRAS-friendly, Alustre products compare very well to current commercial formulations on all measured parameters, despite the absence of some of the objectionable ingredients widespread in the field. This indicates that the safer lacquers proposed in the present invention can be successfully introduced into the market, without having to accept compromised performance.

TEST OF THE ALISTRE PRODUCTS with and without the addition of micron scale diamonds

Test work on the Alustre products has unexpectedly shown that adding micron scale diamonds to the lacquer formulation provides a strengthening effect (evident in hardness measurements), without the need to use expensive and consumer-avoided nanodiamonds (i.e. diamond particles with a dimension of less than 100 nm).

So one can observe from Table 6 below the inventors have found that by adding micro diamonds to the lacquer the mechanical strength (as measured by hardness) is improved, as evident from a comparison of the hardness after 24 hrs for Alustre 607 (with micro diamonds) and Alustre 609 (without diamonds). The hardness is also higher than a comparable commercial product brand designated as Chromavis. All three formulations passed an adhesion test indicating no loss of the applied film.

TABLE 6
Result of comparative gloss, hardness and adhesion tests of
the Alustre formulation with and without infused diamonds.
COLOR NAIL POLISH ADDITIONAL ANALYSIS - selected shade
227 660 (color code) 60 . . . (texture code)

| | | | 227 660 609 |
|---|---|---|---|
| | 227 660 600 | | ALUSTRE |
| | CHROMAVIS | 227 660 607 | texture but |
| | TEXTURE from | ALUSTRE | no diamond in |
| Product name | portfolio | texture | formula |
| Diamond | 0% | 0.05% | 0% |
| content % | | | |
| GLOSS | 74 | 77 | 76 |
| EVALUATION | | | |
| RESULT after | | | |
| 24 h | | | |
| HARDNESS | 160 | 175 | 169 |
| after 24 h | | | |
| ADHESION | 0 | 0 | 0 |
| TEST | | | |
| RESULTS | No loss of | No loss of | No loss of |
| | coating | coating | coating |
| | material | material | material |

The diamond particles used in the 607 Alustre formulation were obtained from a commercial supplier of natural diamonds (IDC Holdings of Hatton Garden, London, UK). The size distribution properties of these particles (obtained from a commercially available Beckman Coulter particle analyzer) are set out in the table below.

TABLE 7
Size and distribution properties of the micron
scale diamond particles of 607 Alustre.

| | | | |
|---|---|---|---|
| Diamond | Diameter | Particle | Particle |
| Particles | measurements | distribution | size (μm) |

| | | | | |
|---|---|---|---|---|
| Mean | 1.916 | μm | D1 | 1.113 |
| Standard | 0.496 | μm | D5 | 1.242 |
| deviation | | | | |
| Median | 1.838 | μm | D10 | 1.332 |
| D (0, 0) | 1.562 | μm | D50 | 1.838 |
| Mode diameter | 1.748 | μm | D95 | 2.864 |
| Specific | 9816 | cm²/g | D95 | 3.308 |
| surface area | | | | |

The diamonds in this batch have a mean diameter of about 2 microns (1.916 pm) with a standard deviation of about 0.5 microns (0.496). The distribution provides an effective upper limit (D99) of 3.308 pm and an effective lower limit (Di) of 1.113 pm. We define the number of particles having diameters above and below the D99 and D1 threshold sizes respectively as being insignificant, i.e. essentially absent.

When we quote a range of particle sizes suitable for use in lacquers in accordance with the invention, we mean that the particles have a mean diameter within the quoted range. In a preferred aspect of the invention the particles should have a diameter distribution so that the mean particle size+/−the standard deviation is within the quoted range.

Most preferably, the Di diameter of the particles is greater than the lower end of the quoted range, and the D99 diameter is lower than the upper end of the quoted range. This ensures that negligible numbers of particles are outside of the target average particle range, so as to provide optimum reasonable strength/hardness properties, without straying into the undesirable nanodiamond range.

In accordance with the invention the particle size (typically diameter) range is preferably greater than 0.10 microns to 10.0 microns, more preferably from 0.2 microns to 5.0 microns. Above 5.0 microns there is a risk that diamond particles will separate on standing, over time. So the most preferable range is from 0.3 microns to 3.0 microns. Sizes below 0.1 microns means (by definition) undesirable nanoparticles. Nanoparticles may of course inevitably be present in practice, but in sufficiently small quantities to be safely ignored.

If the particles are larger than 10 microns, the smooth surface film of the solid lacquer begins to feel gritty and the aesthetic appearance (gloss) is less satisfactory. Hence 10 microns may be set as the upper limit for diamond particle size/diameter. For optimum gloss the present inventors have assessed the benefits of providing diamond particles of less than 10 microns in size, but preferably less than 5.0 microns, and most preferably less than 3.0 microns.

To suspend the diamonds in the liquid formulation two suspending agents (or dispersant) are used: Stearalkonium bentonite and silica. In order to incorporate the diamond powder successfully a pre-mix of: Nitrocellulose+solvents+plasticizer+diamonds is made, which ensures a very efficient process for dispersion of diamonds throughout the liquid formulation and retention of the glossiness of the finished surface. Both suspending agents are GRAS labelled, in line with the aims of the invention. The amount of diamond added was enough to be 0.05 wt % of the final liquid lacquer formulation.

Silica is used as a thickening agent that prevents premature settling of pigments, but in other formulations also lowers the gloss of polish. Silica is often used in nail polish formulations as a thickening agent and as a way to improve the texture of the polish. For this application the silica is in the form of “fumed silica”. Fumed silica is a type of synthetic amorphous silica that is commonly used in cosmetics as a thickening agent, emulsifier, and stabilizer. It is a fine powder that is created by vaporizing silica compounds and then allowing them to cool and condense into a solid form.

Stearalkonium bentonite is a thickening agent that controls liquid flow during application and helps prevent rapid settling of pigments. Stearalkonium bentonite is often used in combination with other ingredients, such as silica, to create the desired texture and consistency for the nail polish.

The results show that both gloss and hardness of the nail polish film are enhanced by the use of micro scale diamonds (with a mean diameter of 1.9 microns), without any loss of film adhesion. Overall, the results show that the GRAS I EPA standard lacquers in accordance with the invention have comparable properties (gloss quality, drying time, and 24 hrs hardness and adhesion) as current commercially available ‘unsafe’ lacquers. Indeed, the diamond-containing version has measurably improved hardness, thought to arise from an increase in film strength due to the dispersed micro-diamonds.

In summary, the present invention relates to the field of human or animal nailcare, and relates in particular to nail lacquers which have safe properties and/or contain a particular distribution of diamond particles. The inventive lacquer is preferably composed of substances generally considered to be safe (also known in the field as GRAS—Generally Regarded As Safe). By lacquer we mean any liquid to be applied to a nail and which sets to form a hard film or layer. The invention provides a liquid lacquer composition for forming a solid cosmetic coating on nail tissue, the composition comprising: one or more film-forming polymers or polymer precursors, one or more solvent for the polymer or polymer precursor, one or more plasticizers which promote flexibility of the solid cosmetic coating and optionally one or more pigments or optical modifiers. The liquid lacquer composition preferably further comprises diamond particles having a mean size within the range of 0.1 micron to 10 microns dispersed within the liquid. These may be provided in amounts from 0.01 to 0.20 wt %.

1-18. (canceled)

19. A liquid lacquer composition for forming a solid cosmetic coating on nail tissue, the composition comprising:
diamond particles having a mean size within the range of 0.1 micron to 10 microns;
one or more film-forming polymers or polymer precursors selected from the group consisting of nitrocellulose, adipic acid/neopentyl glycol/trimellitic anhydride copolymer, acrylates copolymer, and cellulose acetate butyrate; and
one or more solvent for the polymer or polymer precursor selected from the group consisting of ethyl acetate, butyl acetate, ethyl alcohol, isopropyl alcohol, stearalkonium bentonite, acetone, and methyl ethyl ketone (MEK).

20. The liquid lacquer composition according to claim 19 wherein the diamond particles have a mean size within the range of 0.2 to 5.0 microns.

21. The liquid lacquer composition according to claim 19 wherein the diamond particles have a mean size within the range of 0.3 to 3.0 microns.

22. The liquid lacquer composition according to claim 19 wherein the diamond particles have a mean diameter of 1.916 microns with standard deviation 0.496 microns.

23. The liquid lacquer composition according to claim 19 wherein the diamond particles have a diameter from 1.113-3.308 microns.

24. The liquid lacquer composition according to claim 19, wherein the one or more film-forming polymers or polymer precursors is nitrocellulose.

25. The liquid lacquer composition according to claim 19, comprising a dispersant selected from the group consisting of fumed silica, stearalkonium bentonite, and a combination of fumed silica and stearalkonium bentonite.

26. The liquid lacquer composition according to claim 19, wherein the diamond particles are present in an amount of 0.01 to 0.20 wt %.

27. The liquid lacquer composition according to claim 19, wherein the diamond particles are present in an amount of 0.02 to 0.08 wt %.

28. The liquid lacquer composition according to claim 19, wherein the diamond particles are present in an amount of 0.05 wt %.

29. The liquid lacquer composition according to claim 19, comprising a plasticizer selected from one or more of: acetyl tributyl citrate, triethyl citrate, and sucrose benzoate.

30. The liquid lacquer composition according to claim 19, comprising a pigment is selected from one or more of: silica, citric acid, ferric ammonium ferrocyanide, D & C Red #6 Barium Lake, D & C Red #7 Calcium Lake, FD & C Yellow #5 Aluminum Lake, Cl 77891, Cl 15880, aluminium hydroxide, tin oxide, Cl 77499, Cl 77007, Cl 77742, CI12085, Cl 42090, Cl 77000, Cl 60725.

31. The liquid lacquer composition according to claim 19, wherein the composition is a base coat comprising:
wherein the diamond particles are present in an amount of 0.01 to 0.20 wt %;
one or more film-forming polymers or polymer precursors in amounts comprising nitrocellulose <11 wt %, copolymer of adipic acid/neopentyl glycol/trimellitic anhydride <5.3 wt %, and acrylates copolymer <4 wt %;
a plasticizer in amounts comprising acetyl tributyl citrate <5 wt % and triethyl citrate <5 wt %;
a pigment in amounts comprising silica <0.4 wt %, citric acid <0.0001 wt %, D&C Red #6 Barium Lake <0.005 wt %, CI 77891<0.08 wt %, and aluminum hydroxide <0.1 wt %;
and the one or more solvent in amounts comprising ethyl acetate <38 wt %, butyl acetate <28 wt %, ethyl alcohol <6 wt %, isopropyl alcohol <0.2 wt %, and stearalkonium bentonite <1.5 wt %;
all percentages totaling 100%.

32. The liquid lacquer composition according to claim 19, wherein the composition is a nail polish comprising:
one or more film-forming polymers or polymer precursors in amounts comprising nitrocellulose <12 wt %, copolymer of adipic acid/neopentyl glycol/trimellitic anhydride <5.3 wt %, and acrylates copolymer <4 wt %;
a plasticizer comprising acetyl tributyl citrate <5 wt %, triethyl citrate <5 wt %, and sucrose benzoate <0.2 wt %;
a pigment comprising silica <0.65 wt %, citric acid <0.0001 wt %, ferric ammonium ferrocyanide <1 wt %, D&C Red #6 Barium Lake <1.3 wt %, D&C Red #7 Calcium Lake <0.6 wt %, FD&C Yellow #5 Aluminum Lake <1.4 wt %, CI 77891<2.7 wt %, CI 15880<0.8 wt %, aluminum hydroxide <0.1 wt %, tin oxide <0.2 wt %, CI 77499<3.0 wt %, CI 77007<3.0 wt %, CI 77742<1.0 wt %, CI 12085<0.8 wt %, CI 42090<0.5 wt %, CI 77000<0.06 wt %, CI 60725<0.0002 wt %;
and the solvent comprising ethyl acetate <42 wt %, butyl acetate <31 wt %, ethyl alcohol <6 wt %, isopropyl alcohol <0.15 wt %, stearalkonium bentonite <1.7 wt %, acetone <0.2 wt %, and methyl ethyl ketone <0.01 wt %;
all percentages totaling 100%.

33. The liquid lacquer composition according to claim 19, wherein the composition is a top coat comprising:
one or more film-forming polymers or polymer precursors in amounts comprising nitrocellulose <8.5 wt %, acrylates copolymer <4 wt %, and cellulose acetate butyrate <3 wt %;
a plasticizer comprising acetyl tributyl citrate <5 wt %, and triethyl citrate <5 wt %;
and the solvent comprising ethyl acetate <36 wt %, butyl acetate <35 wt %, ethyl alcohol <5 wt %, and methyl ethyl ketone <0.01 wt %;
all percentages totaling 100%.

34. The nail polish according to claim 32, wherein the composition after a drying time ranging from 2′20″-3′30″ has a hardness after 24 h of at least 176.

35. The top coat according to claim 33, wherein the composition after a drying time ranging from 1′54″-2′24″ has a hardness after 24 h of at least 125.

36. The liquid lacquer composition according to claim 19, wherein the polymer/precursor excludes tosylamide/formaldehyde resin (TSF Resin).

37. The liquid lacquer composition according to claim 29, wherein the plasticizer excludes dibutyl phthalate (DBP), trimethyl pentanyl diisobutyrate, triphenyl phosphate, and camphor.

38. The liquid lacquer composition according to claim 30, wherein the pigment excludes mica, titanium dioxide, bismuth oxychloride, Cl 777266.

39. The liquid lacquer composition according to claim 19, wherein the one or more solvent excludes propyl acetate, toluene, benzophenone-1, and dimethicone.
